(19) United States
(12) Patent Application Publication
(10) Pub. No.: US 2023/0211094 A1
(43) Pub. Date: Jul. 6, 2023

(54) MAIN UNIT AND ELECTRONIC VAPORIZATION DEVICE

(71) Applicant: Jiangmen Moore Technology., Ltd, Jiangmen (CN)

(72) Inventor: Huakai YUAN, Jiangmen (CN)

(21) Appl. No.: 18/087,010

(22) Filed: Dec. 22, 2022

(30) Foreign Application Priority Data
Dec. 30, 2021 (CN) 202111651880.5

(51) Int. Cl. A61M 11/04

(52) U.S. Cl. CPC A61M 11/042; A61M 2205/8212; A61M 2205/502; A61M 2205/3368

(57) ABSTRACT

A main unit connected to a vaporizer and controlling operation of the vaporizer includes: a processor; and a battery, a timer, and a memory that are connected to the processor. The processor controls the battery to provide energy for the vaporizer to allow the vaporizer to vaporize an aerosol-generating substrate. The processor calculates an accumulative temperature during vaporization of the vaporizer based on timing information of the timer and a vaporization temperature change curve stored in the memory.

### CROSS-REFERENCE TO PRIOR APPLICATION

Priority is claimed to Chinese Patent Application No. 202111651880.5, filed on Dec. 30, 2021, the entire disclosure of which is hereby incorporated by reference herein.

### FIELD

This application relates to the field of electronic vaporization technologies, and in particular, to a main unit and an electronic vaporization device.

### BACKGROUND

An electronic vaporization device is composed of a vaporizer and a main unit. The vaporizer is used to store and vaporize an aerosol-generating substrate, and the main unit is used to provide energy for vaporization of the vaporizer and control the vaporizer to vaporize the aerosol-generating substrate.

In the existing electronic vaporization device, a temperature measurement function of the vaporizer is implemented by using various sensors. In addition, problems such as high-frequency continuous inhalations and irregular self-starting due to microphone failure cause heat to accumulate in the vaporizer and gradually exceed the temperature resistance of the vaporizer, which further leads to melting or deformation of the vaporizer, causing problems such as leakage.

At present, most electronic vaporization devices are provided with protection for continuous inhalation that lasts a certain period of time, but do not consider the impact of the heat accumulation in a plurality of inhalations on the vaporizer.

### SUMMARY

In an embodiment, the present invention provides a main unit connected to a vaporizer and controlling operation of the vaporizer, the main unit comprising: a processor; and a battery, a timer, and a memory that are connected to the processor, wherein the processor is configured to control the battery to provide energy for the vaporizer to allow the vaporizer to vaporize an aerosol-generating substrate, wherein the processor is configured to calculate an accumulative temperature during vaporization of the vaporizer based on timing information of the timer and a vaporization temperature change curve stored in the memory.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an embodiment of an electronic vaporization device provided in this application;

FIG. 2 is a schematic structural diagram of an embodiment of a main unit provided in this application;

FIG. 3 is a diagram of a vaporization temperature change curve of a vaporizer provided in an embodiment of this application;

FIG. 4 is a schematic structural diagram of another embodiment of a main unit provided in this application; and

FIG. 5 is a schematic flowchart of a working process of a processor provided in this application.

### DETAILED DESCRIPTION

In an embodiment, the present invention provides a main unit and an electronic vaporization device to resolve a technical problem of how to detect heat accumulation during vaporization of a vaporizer in the prior art.

In an embodiment, the present invention provides: a main unit connected to a vaporizer and controlling operation of the vaporizer is provided, the main unit including: a processor, and a battery, a timer, and a memory that are connected to the processor; the processor controls the battery to provide energy for the vaporizer to allow the vaporizer to vaporize an aerosol-generating substrate, and the processor further calculates an accumulative temperature during vaporization of the vaporizer based on timing information of the timer and a vaporization temperature change curve stored in the memory.

The main unit further includes an airflow sensor, and the processor is connected to the airflow sensor and the timer to obtain a start time and an end time for each inhalation of the vaporizer.

The vaporization temperature change curve is a temperature change curve for one inhalation of the vaporizer, and the processor calculates the accumulative temperature during vaporization of the vaporizer based on the vaporization temperature change curve, and the start time and the end time for each inhalation of the vaporizer.

The accumulative temperature obtained by the processor during vaporization of the vaporizer includes an accumulative increased temperature based on the vaporization temperature change curve during an inhalation, and an accumulative decreased temperature based on the vaporization temperature change curve during an inhalation interval.

The processor obtains an interval duration between two adjacent inhalations of the vaporizer through the airflow sensor and the timer; and the processor controls the timer to reset in response to the interval duration being longer than a first preset duration, and restarts the calculation of the accumulative temperature during vaporization of the vaporizer.

A plurality of vaporization temperature change curves are stored in the memory, and the processor further obtains a parameter of the vaporizer, and obtains the vaporization temperature change curve corresponding to the vaporizer based on the parameter of the vaporizer.

The main unit further includes a prompter connected to the processor; the processor controls the prompter to send a first prompt message in response to the accumulative temperature during vaporization of the vaporizer being higher than a preset temperature.

The processor controls the prompter to send the first prompt message, and controls the battery to stop providing energy for the vaporizer at the same time.

The main unit further includes a detector connected to the processor; the processor controls the prompter to stop sending the first prompt message in response to detecting a reset signal of the main unit by the detector.

The processor controls the battery to stop providing energy for the vaporizer in response to duration of the first prompt message sent by the processor being longer than a second preset duration, and controls the prompter to send a second prompt message.

The processor controls the timer to reset in response to the accumulative temperature during the vaporization of the vaporizer being not higher than a room temperature, and restarts the calculation of the accumulative temperature during vaporization of the vaporizer.

To resolve the technical problems, a second technical solution provided in this application is as follows: an electronic vaporization device is provided, including: a vaporizer and a main unit described above. This application provides a main unit and an electronic vaporization device. The main unit includes a processor, and a battery, a timer, and a memory that are connected to the processor; the processor controls the battery to provide energy for the vaporizer to vaporize an aerosol-generating substrate, and the processor also obtains an accumulative temperature during vaporization of the vaporizer based on timing information of the timer and a vaporization temperature change curve stored in the memory. Through the foregoing setting, there is no need to arrange a temperature sensor on the vaporizer. The accumulative temperature during vaporization of the vaporizer can be obtained according to the timer and the vaporization temperature change curve stored in the memory, and heat accumulation of the vaporizer can be obtained based on the accumulative temperature during the vaporization of the vaporizer.

The technical solutions in the embodiments of this application are clearly and completely described below with reference to the accompanying drawings in the embodiments of this application. Apparently, the described embodiments are merely some rather than all of the embodiments of this application. Based on the embodiments of this application, all other embodiments obtained by a person of ordinary skill in the art without creative efforts shall fall within the protection scope of this application.

In the following description, for the purpose of illustration rather than limitation, specific details such as the specific system structure, interface, and technology are proposed to thoroughly understand this application.

The terms “first”, “second”, and “third” in this application are merely intended for a purpose of description, and shall not be understood as indicating or implying relative significance or implicitly indicating the number of indicated technical features. Therefore, features defining “first”, “second”, and “third” can explicitly or implicitly include at least one of the features. In the description of this application, “a plurality of” means at least two, such as two and three unless it is specifically defined otherwise. All directional indications (for example, upper, lower, left, right, front, and back) in the embodiments of this application are only used for explaining relative position relationships, movement situations, or the like among the various components in a specific posture (as shown in the accompanying drawings). If the specific posture changes, the directional indications change accordingly. In the embodiments of this application, the terms “include”, “have”, and any variant thereof are intended to cover a non-exclusive inclusion. For example, a process, method, system, product, or device that includes a series of steps or units is not limited to the listed steps or units, but further optionally includes a step or unit that is not listed, or further optionally includes another step or component that is intrinsic to the process, method, product, or device.

“Embodiment” mentioned in this specification means that particular features, structures, or characteristics described with reference to the embodiment may be included in at least one embodiment of this application. The term appearing at different positions of this specification may not refer to the same embodiment or an independent or alternative embodiment that is mutually exclusive with another embodiment. A person skilled in the art explicitly or implicitly understands that the embodiments described in this specification may be combined with other embodiments.

This application is further described in detail below with reference to the accompanying drawings and embodiments.

Referring to FIG. 1, FIG. 1 is a schematic structural diagram of an embodiment of an electronic vaporization device according to this application. In this embodiment, an electronic vaporization device 100 is provided. The electronic vaporization device 100 may be configured to vaporize an aerosol-generating substrate. The electronic vaporization device 100 includes a vaporizer 1 and a main unit 2 that are electrically connected to each other.

The vaporizer 1 is configured to store the aerosol-generating substrate and vaporize the aerosol-generating substrate to form aerosols that can be inhaled by a user. The vaporizer 1 may be specifically used in different fields, for example, medical treatment, cosmetics, and leisure smoking. In a specific embodiment, the vaporizer 1 may be used for an electronic aerosolization device, to generate an inhalable aerosol from an aerosol-generating substrate. The following embodiments are examples of leisure smoking. Of course, in other embodiments, the vaporizer 1 may further be used for a hair spraying device to vaporize a hair spray for hair styling, or used for a device for treating upper and lower respiratory diseases to vaporize medical drugs. For the specific structure and function of the vaporizer 1, reference may be made to the specific structure and function of the vaporizer 1 in any of the following embodiments, and can achieve the same or similar technical effects. Details are not described herein again. A main unit 2 provides energy for the vaporizer 1 to vaporize an aerosol-generating substrate, and controls the vaporizer 1 to vaporize the aerosol-generating substrate.

The vaporizer 1 and the main unit 2 may be integrally provided or detachably connected, and may be designed based on specific needs.

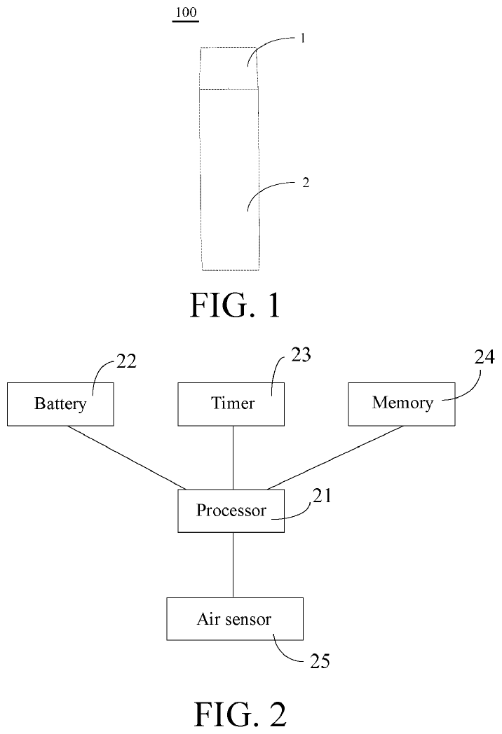

Referring to FIG. 2, FIG. 2 is a schematic structural diagram of an embodiment of a main unit provided in this application. The main unit 2 includes a processor 21, a battery 22, a timer 23, and a memory 24.

The processor 21 controls the battery 22 to provide energy for a vaporizer 1 to allow the vaporizer 1 to vaporize an aerosol-generating substrate; the timer 23 is configured to collect timing information during inhalation of the vaporizer 1, and the memory 24 stores a vaporization temperature change curve. The processor 21 calculates a cumulative temperature during vaporization of the vaporizer 1 based on the timing information of the timer 23 and the vaporization temperature change curve stored in the memory 24, so as to obtain heat accumulation of the vaporizer 1.

The main unit 2 further includes an airflow sensor 25, and the airflow sensor 25 is configured to detect an inhaling signal of the vaporizer 1. In one embodiment, the airflow sensor 25 is a microphone. The processor 21 is connected to the airflow sensor 25. The processor 21 obtains inhalation information of the vaporizer 1 based on the airflow sensor 25, and then in combination with the timing function of the timer 23, a start time and an end time of each inhalation of the vaporizer 1, and an interval between two adjacent inhalations can be obtained. It may be understood that one inhalation is commonly referred to as one draw, and the interval between two adjacent inhalations is commonly referred to as an interval between two draws.

In this embodiment, the vaporization temperature change curve stored in the memory 24 is a temperature change curve for one inhalation of the vaporizer 1, and the processor 21 calculates a cumulative temperature during vaporization of the vaporizer based on the vaporization temperature change curve, and the start time and the end time for each inhalation of the vaporizer. The cumulative temperature during vaporization of the vaporizer 1 obtained by the processor 21 includes an accumulative increased temperature obtained from the vaporization temperature change curve during an inhalation, and an accumulative decreased temperature obtained from the vaporization temperature change curve during an inhalation interval.

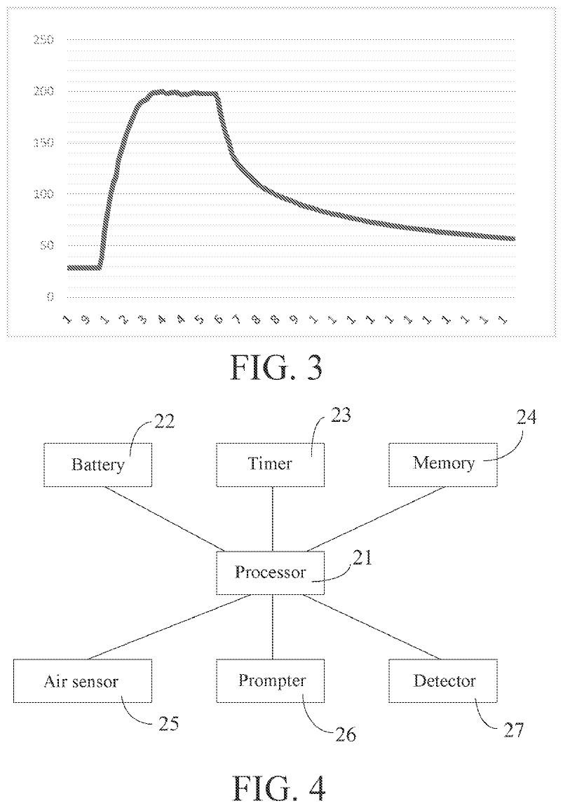

Exemplarily, referring to FIG. 3, FIG. 3 is a diagram of vaporization temperature change curve of a vaporizer provided in an embodiment of this application. As shown in FIG. 3, the temperature of the vaporizer 1 rises for a few seconds at the beginning of each inhalation. For example, the temperature rises from the 15th second to the 29th second (the heating up time is 14 seconds). After the temperature reaches a vaporization temperature of the aerosol-generating substrate, the temperature is maintained for vaporization. For example, the temperature is maintained from the 29th second to the 64th second; when the inhalation stops, that is, during the interval between the current inhalation and the next inhalation, the temperature of the vaporizer 1 begins to decrease. For example, the temperature decreases after the 64th second.

For the first inhalation, the start time is M and the end time is N. The duration of the first inhalation is a difference between the end time N and the start time M, and the difference is not less than 14 seconds. At the beginning of the first inhalation, the temperature of the vaporizer 1 is room temperature; at the time N, the temperature of the vaporizer 1 is the vaporization temperature of the aerosol-generating substrate. The temperature of the vaporizer 1 rises from the room temperature to the vaporization temperature of the aerosol-generating substrate, and an accumulative increased temperature A1 is obtained based on the vaporization temperature change curve shown in FIG. 3.

For the second inhalation, the start time is S and the end time is T. The duration of the second inhalation is a difference between the end time T and the start time S, and the difference is not less than 14 seconds. An interval between the second inhalation and the first inhalation is a difference between the time S and the time N. During the time interval, the temperature of vaporizer 1 decreases. The temperature of the vaporizer 1 decreases from the vaporization temperature of the aerosol-generating substrate to R, and the second inhalation is started. An accumulative decreased temperature B1 is obtained based on the vaporization temperature change curve shown in FIG. 3. At the time S, the temperature of the vaporizer 1 is R; in the process from the time S to the time T, the temperature of the vaporizer 1 is increased by the temperature A1 cumulatively on the basis of the temperature R. At the time T, the temperature of the vaporizer 1 is not lower than the vaporization temperature of the aerosol-generating substrate.

The first accumulative increased temperature A1, the accumulative decreased temperature B1, and the second accumulative increased temperature A1 are summed to obtain the accumulative temperature during vaporization of the vaporizer 1 from the beginning of the first inhalation to the end of the second inhalation. It may be understood that as the interval between the second inhalation and the first inhalation decreases, the accumulative decreased temperature B1 during this period is lower, and at the time T, the temperature of the vaporizer 1 is higher.

In other words, during inhalation of the vaporizer 1, the processor 21 performs a simulated warming process based on the vaporization temperature change curve pre-stored in the memory 24, and the temperature of the vaporizer 1 increases by a fixed value based on a fixed time. During the inhalation interval of the vaporizer 1, the processor 21 performs a simulated cooling process based on the vaporization temperature change curve pre-stored in the memory 24, and the temperature of the vaporizer 1 decreases by a fixed value based on a fixed time.

When the interval between two adjacent inhalations of the vaporizer 1 obtained by the processor 21 through the airflow sensor 25 and the timer 23 is longer than the first preset duration, the processor 21 controls the timer 23 to reset and restarts the calculation of the accumulative temperature during vaporization of the vaporizer 1. The first preset duration is longer than the duration required for decreasing the temperature of the vaporizer 1 from the vaporization temperature of the aerosol-generating substrate to the room temperature. It may be understood that when the interval between two adjacent inhalations of the vaporizer 1 is longer than the first preset duration, it indicates that the user is not using the vaporizer 1 temporarily, and the calculation of the accumulative temperature during vaporization of the vaporizer 1 is started again when the vaporizer 1 is used next time.

In an embodiment, a plurality of vaporization temperature change curves are stored in the memory 24, and the processor 21 is further configured to obtain a parameter of the vaporizer 1 and obtain a vaporization temperature change curve corresponding to the vaporizer 1 based on the parameter of the vaporizer 1, to calculate the cumulative temperature during vaporization of the vaporizer 1. It may be understood that different vaporizers may have different vaporization temperature change curves, and it helps improve the accuracy of calculation by selecting the vaporization temperature change curve corresponding to the vaporizer 1 to calculate the accumulative temperature during vaporization of the vaporizer 1.

The memory 24 stores at least one vaporization temperature change curve, which can be specifically designed based on the requirements for the accuracy of overheating protection of the vaporizer 1.

Referring to FIG. 4, FIG. 4 is a schematic structural diagram of another embodiment of a main unit provided in this application. The processor 21 controls the timer 23 to reset in response to the accumulative temperature during vaporization of the vaporizer 1 being not higher than the room temperature, and restarts calculation of the accumulative temperature during vaporization of the vaporizer 1; the processor 21 performs corresponding processing in response to the accumulative temperature during vaporization of the vaporizer 1 being higher than a preset temperature, to prevent the vaporizer 1 from exceeding temperature resistance of the vaporizer 1, thereby achieving overheating protection for the vaporizer 1.

Furthermore, the main unit 2 further includes a prompter 26. The prompter 26 is connected to the processor 21.

The processor 21 controls the prompter 26 to send a first prompt message in response to the accumulative temperature during vaporization of the vaporizer 1 being higher than a preset temperature. The processor 21 sends the first prompt message to remind a user of the risk of overheating of the vaporizer 1 and inform the user to perform a related operation. For example, when the user receives the first prompt message, the user may reset the main unit 2 by plugging the vaporizer 1 or by charging the vaporizer 1. It may be understood that, this application achieves the over-temperature alarm of the vaporizer 1 without using a temperature sensor.

The main unit 2 further includes a detector 27, and the detector 27 is connected to the processor 21; the processor 21 controls the prompter 26 to stop sending the first prompt message in response to detecting a reset signal of the main unit 2 by the detector 27.

Optionally, the processor 21 controls the prompt 26 to send the first prompt message and controls the battery 22 to stop provide energy for the vaporizer 1 at the same time, to avoid overheating of the vaporizer 1, so as to achieve overheating protection.

Optionally, the processor 21 controls the battery 22 to provide energy for the vaporizer 1 in response to duration of the first prompt message sent by the prompter 26 being longer than a second preset duration, and controls the prompter to send a second prompt message. When the duration of the first prompt message is longer than the second preset duration, and a user has not performed a related operation to reset the main unit 2, the battery 22 is stopped from providing energy, so as to prevent the vaporizer 1 from being overheated, thus achieving the overheating protection. At the same time, a second prompt message is sent to remind the user to reset the main unit 2.

It may be understood that the first prompt message and the second prompt message may be both sound and light prompts. In an embodiment, the first prompt message is the same as the second prompt message. In an embodiment, the first prompt message is different from the second prompt message, and prompt intensity of the second prompt message is higher than that of the first prompt message. For example, the first prompt message is a flashing red light, and the second prompt message is a red light that is constantly on.

Through the foregoing setting, there is no need to arrange various sensors on the vaporizer 1 for temperature measurement, and there is no need to arrange a temperature measurement circuit on the main unit 2. The real-time temperature of the vaporizer 1 can be obtained from the timing information of the timer 23 and the vaporization temperature change curve stored in the memory 24, and then the accumulative temperature during vaporization of the vaporizer 1 can be obtained. In other words, the operation of increasing and decreasing the temperature of the vaporizer 1 is accomplished in a simple manner by setting a virtual temperature variable on the processor 21 of the main unit 2 based on the timing information of the timer 23 and the vaporization temperature change curve stored in the memory 24. Considering the accumulative temperature during the vaporization of the vaporizer 1, the temperature of the vaporizer 1 can be prevented from exceeding temperature resistance of the vaporizer 1, thereby achieving overheating protection.

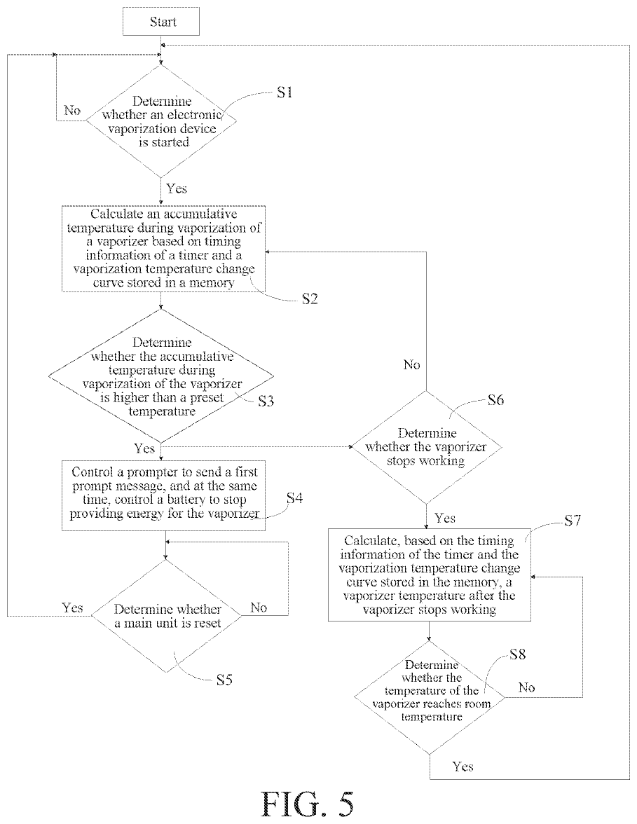

Referring to FIG. 5, FIG. 5 is a schematic flowchart of a working process of a processor provided in this application. The processor 21 implements the overheating protection process for the vaporizer 1 through the following specific steps:

Steps S1: Determine whether an electronic vaporization device is started.

Specifically, when the processor 21 obtains an inhalation signal through the airflow sensor 25, the electronic vaporization device is started, and step S2 is performed; otherwise, step S1 is continued.

Step S2: Calculate an accumulative temperature during vaporization of a vaporizer based on timing information of a timer and a vaporization temperature change curve stored in a memory.

Specifically, the processor 21 obtains inhalation information of the vaporizer 1 based on the airflow sensor 25, and in combination with the timing function of the timer 23, a start time and an end time of each inhalation of the vaporizer 1, and an interval between two adjacent inhalations can be obtained.

In this embodiment, the vaporization temperature change curve stored in the memory 24 is a temperature change curve for one inhalation of the vaporizer 1, and the processor 21 calculates an accumulative temperature during vaporization of the vaporizer 1 based on the vaporization temperature change curve, and the start time and the end time for each inhalation of the vaporizer 1. The accumulative temperature during vaporization of the vaporizer 1 obtained by the processor 21 includes an accumulative increased temperature obtained from the vaporization temperature change curve during inhalation, and an accumulative decreased temperature obtained from the vaporization temperature change curve during an inhalation interval.

Step S3: Determine whether the accumulative temperature during vaporization of the vaporizer is higher than a preset temperature. If the accumulative temperature during vaporization of the vaporizer 1 is higher than the preset temperature, step S4 is performed; if the accumulative temperature during vaporization of the vaporizer 1 is not higher than the preset temperature, step S6 is performed.

Step S4: Control a prompter to send a first prompt message, and at the same time, control a battery to stop providing energy for the vaporizer.

By controlling the battery to stop providing energy for the vaporizer 1, it prevents the temperature of the vaporizer 1 is from becoming excessively high; the prompter 26 is controlled to send the first prompt message to remind a user of the risk of overheating of the vaporizer 1, and inform the user to perform a related operation. For example, when the user receives the first prompt message, the user may reset the main unit 2 by plugging the vaporizer 1 or by charging the vaporizer 1.

Step S5: Determine whether a main unit is reset.

Specifically, the detector 27 is used to detect whether the main unit 2 is reset; if the main unit 2 is reset, the prompter 26 is controlled to stop sending the first prompt message, and step S1 is performed; otherwise, step S5 is continued.

Step S6: Determine whether the vaporizer stops working.

If the vaporizer 1 does not stop working, step S2 is performed; if the vaporizer 1 stops working, step S7 is performed.

Step S7: Calculate, based on the timing information of the timer and the vaporization temperature change curve stored in the memory, a vaporizer temperature after the vaporizer stops working.

Step S8: Determine whether the temperature of the vaporizer reaches room temperature.

If the temperature of the vaporizer 1 decreases to room temperature after the vaporizer 1 stops working, step S1 is performed; if the temperature of the vaporizer 1 is higher than room temperature after the vaporizer 1 stops working, step S7 is performed.

In the process of implementing overheating protection for the vaporizer 1 in this application, there is no need to set various sensors on the vaporizer 1 for temperature measurement. The real-time temperature of the vaporizer 1 can be obtained from the timing information of the timer 23 and the vaporization temperature change curve stored in the memory 24, and the accumulative temperature during vaporization of the vaporizer 1 can be obtained, so as to obtain heat accumulation of the vaporizer 1. In other words, the operation of increasing and decreasing the temperature of the vaporizer 1 is accomplished in a simple manner by setting a virtual temperature variable on the processor 21 based on the timing information of the timer 23 and the vaporization temperature change curve stored in the memory 24.

The descriptions are merely implementations of this application, and the patent scope of this application is not limited thereto. All equivalent structure or process changes made according to the content of this specification and accompanying drawings in this application or by directly or indirectly applying this application in other related technical fields shall fall within the protection scope of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A main unit connected to a vaporizer and controlling operation of the vaporizer, the main unit comprising:
a processor; and
a battery, a timer, and a memory that are connected to the processor,
wherein the processor is configured to control the battery to provide energy for the vaporizer to allow the vaporizer to vaporize an aerosol-generating substrate,
wherein the processor is configured to calculate an accumulative temperature during vaporization of the vaporizer based on timing information of the timer and a vaporization temperature change curve stored in the memory.

2. The main unit of claim 1, further comprising:
an airflow sensor,
wherein the processor is connected to the airflow sensor and the timer to obtain a start time and an end time for each inhalation of the vaporizer.

3. The main unit of claim 2, wherein the vaporization temperature change curve comprises a temperature change curve for one inhalation of the vaporizer,
wherein the processor is configured to calculate the accumulative temperature during vaporization of the vaporizer based on the vaporization temperature change curve and the start time and the end time for each inhalation of the vaporizer.

4. The main unit of claim 3, wherein the cumulative temperature obtained by the processor during vaporization of the vaporizer comprises an accumulative increased temperature based on the vaporization temperature change curve during an inhalation and an accumulative decreased temperature based on the vaporization temperature change curve during an inhalation interval.

5. The main unit of claim 2, wherein the processor is configured to obtain an interval duration between two adjacent inhalations of the vaporizer through the airflow sensor and the timer,
wherein the processor is configured to control the timer to reset in response to the interval duration being longer than a first preset duration, and restart a calculation of the accumulative temperature during vaporization of the vaporizer.

6. The main unit of claim 1, wherein a plurality of vaporization temperature change curves are stored in the memory, and
wherein the processor is configured to obtain a parameter of the vaporizer and obtain the vaporization temperature change curve corresponding to the vaporizer based on the parameter of the vaporizer.

7. The main unit of claim 1, further comprising:
a prompter connected to the processor,
wherein the processor is configured to control the prompter to send a first prompt message in response to the accumulative temperature during vaporization of the vaporizer being higher than a preset temperature.

8. The main unit of claim 7, wherein the processor is configured to control the prompter to send the first prompt message and control the battery to stop providing energy for the vaporizer at a same time.

9. The main unit of claim 7, further comprising:
a detector connected to the processor,
wherein the processor is configured to control the prompter to stop sending the first prompt message in response to detecting a reset signal of the main unit by the detector.

10. The main unit of claim 7, wherein the processor is configured to control the battery to stop providing energy for the vaporizer in response to a duration of the first prompt message sent by the prompter being longer than a second preset duration, and to control the prompter to send a second prompt message.

11. The main unit of claim 1, wherein the processor is configured to control the timer to reset in response to the accumulative temperature during vaporization of the vaporizer being not higher than a room temperature, and to restart a calculation of the accumulative temperature during vaporization of the vaporizer.

12. An electronic vaporization device, comprising:
a vaporizer; and
the main unit of claim 1.
